(19) United States
(12) Patent Application Publication
(10) Pub. No.: US 2003/0215354 A1
(43) Pub. Date: Nov. 20, 2003

(54) Systems and methods for in situ soil sterilization, insect extermination and weed killing

(75) Inventors: William J. Clark, Manteca, CA (US); Charles W. Kissell, Fresno, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Advanced Scientific Technologies Corporation, Fresno, CA

(21) Appl. No.: 10/245,787

(22) Filed: Sep. 16, 2002

Related U.S. Application Data
(60) Provisional application No. 60/381,865, filed on May 17, 2002.

(51) Int. Cl. A61L 2/00; B01J 19/00

(52) U.S. Cl. 422/22; 422/38; 422/40; 250/455.11

(57) ABSTRACT

Systems, methods and devices for in situ soil sterilization using contained microwave energy, and more specifically methods and devices for removing from the soil to a specified depth in situ substantially all biological pests. The method comprises exposing the specified depth of soil to sufficient energy flux, within a sufficiently short exposure time, of microwave frequency electromagnetic radiation at a frequency tuned to dissociate biopolymers. The device comprises: a source of microwave frequency electromagnetic radiation; a carriage; and, optionally a radiation shield and a cooling system. The carriage moves the source of electromagnetic radiation to an area for treatment and the radiation shield prevents leakage of radiation to undesired points. The cooling system maintains the device at a sufficiently cool temperature for reliable operation.

### CROSS-REFERENCES TO RELATED APPLICATIONS

This application claims the benefit of U.S. Provisional Serial No. 60/381,865, filed May 17, 2002, which is hereby incorporated by reference in its entirety.

### BACKGROUND OF THE INVENTION

The present invention relates generally to methods for sterilizing soils and to the equipment related thereto.

Many pests, including eukaryotic, prokaryotic and viral pathogens, are known to reduce both agricultural yield and quality of crops, causing considerable economic losses. The pests include for example insect eggs, larvae, nematodes, and weed competitors of crop plants. Soil-born microbes, insects and seeds of undesired plants are especially problematic because treatments that provide desired levels of control are often toxic to the agriculturally desirable plant. Some treatments, such as methyl bromide fumigation, may be environmentally detrimental.

Increasing crop specialization and single-crop systems, particularly widespread in the sector of ornamental plants, row crops, tree crops, vineyards, root crops, etc., perturb or insult the soil microflora because of a predominating effect of the root system of the agriculturally cultivated plants. Additionally, numerous vegetable and animal parasites can survive in the soil for many years, until they come in contact with hosts susceptible to pathology or that serve as vectors to susceptible organisms. They can be particularly problematic for repeat crops when a plant is susceptible to a particular parasite. Repeat cropping with the same crop plants in the same soil typically leads to microflora alteration and parasite accumulation which causes a progressive deterioration in harvest yields with successive crops. A long practiced method to ameliorate this problem is the use of “crop rotation” in which the interval that a crop remains absent from a soil locale is extended to permit microflora to more fully return to a pre-perturbation ecological balance, and to deplete soil dwelling parasites of the desired crop plant by reducing availability of suitable host plants. However, often, as for example with melons, rotation does not provide sufficient reliability because many parasites are able to survive as parasites of other plants or remain in a dormant or metabolically inactive state. Abandoning a certain crop for a sufficient period to reduce the parasite burden in the soil often may be economically impracticable.

When crop rotation or leaving a plot fallow is inadequate or impracticable, a soil locale must be treated to maintain or restore productivity of a certain crop. Known soil treatments for disinfection and de-infestation include chemical or physical killing methods, which are typically time and labor intensive. Both chemical and heat based soil treatments require some form of mechanical penetration, as by pressurized gas or fluid jets, nozzle penetration, mixing or tilling or the like. Not all such mechanical manipulations, for example tilling can, or should, be practiced beyond a certain depth, for example tilling to six feet would cause topsoil to be mixed with underlying strata. Depending upon the specific treatment and the method of obtaining soil penetration, in situ soil sterilization may become economically impracticable for all but the most profitable crops.

Chemical interventions typically include administration of fungicides, insecticides, plant protection products or fumigation products to the soil and/or to the crops. In addition to being time consuming, such treatments are costly and have a high potential for environmental or ecological damage. After chemical treatment of the soil, relatively long time intervals are required before cultivation and/or planting of the treated soil is possible.

Physical interventions presently employed include administering heat to the soil. The heat is produced and distributed in various ways, and regardless of the heat generation method used, heating the soil up to 80-90° C. is sufficient to kill the majority of pests present. This type of operation permits cultivation a short time following treatment, generally as soon as the temperature of the soil drops to below about 25-30° C. Of the heat based treatments, treatments with dry heat are the least effective, often yielding unsatisfactory results, and are difficult to apply to relatively larger plots of land.

Sterilization with boiling water is expensive and generally only economically practicable under limited circumstances. An example of a tool for the in situ disinfection of soil by injection of hot water, disclosed in U.S. Pat. No. 5,622,123, utilizes hot water killing with soil mixing or tilling for penetration and an insulating foam layer which is placed on the soil surface to retain the heat from the water. Some hot water killing methods employ a movable chamber or tent-like hood for penetration to about 10 inches, and a rake or comb-like linear array of nozzles to inject superheated water into the soil for deeper penetration. Steam treatment typically finds applications for soil disinfection in greenhouses, stable seedbeds or small open plots: it can be accomplished by removing the soil to be treated to large cement tanks, or other appropriate containers, then passing the steam through the soil using fixed or movable tools positioned on or within the soil to be disinfected. Typically for steam based methods, steam is generated and moved through pipes to the dispensing tools. Steam and hot water based treatments require both a mechanically complex apparatus to effect penetration, and long hoses between the water heating element and the apparatus for penetrating application to the soil.

Recent approaches to heat based treatment have employed dispersion into the soil of at least one compound, solid, liquid or gaseous, able to react exothermically with water and/or steam, or another substance combined with injection of a jet of water and/or steam, or of another substance, into the soil to produce heat in the subsequent reaction with the compound (see for example U.S. Pat. Nos. 6,319,463 and 6,183,532). An analogous method for generation of microbicidal chemical species in the soil uses treatment with an aqueous solution of an activated oxygen species after pretreatment with a water soluble phenolic complex including a divalent cation having redox potential, a cation redox reducing agent for 40% or greater reduction of soil microorganisms. This method reportedly does not kill seed (see U.S. Pat. No. 5,607,856). An excess of compounds that react exothermically with a chemical species which may be depleted and later replenished in the soil can create a residual problem; similarly an excess of a reagent capable of generating toxic chemical species in the presence of a second reagent can potentially result in toxic residue post treatment.

Therefore, it is desirable to provide methods and devices for effectively and efficiently treating soil to reduce or eliminate agricultural pests including undesirable microorganisms, seeds and plants from an area intended for cultivation of crops. 25

### BRIEF SUMMARY OF THE INVENTION

The present invention provides devices, systems and methods for soil sterilization using contained microwave energy. The methods for treating the soil include exposing a 30 specified depth of soil to a sufficient energy flux of microwave frequency electromagnetic radiation at a specific frequency or range of frequencies, e.g., tuned to dissociate biopolymers, for a sufficient period of time to kill substantially all pests in the area of treatment. Following treatment, the soil may optionally be reconstituted with beneficial organisms to provide a desired soil microbial balance by inoculating the area of treated soil with one or more microbes to produce an inoculated area of treated soil. A device according to the present invention typically includes a source that generates microwave electromagnetic radiation at a frequency tuned to disrupt one or more biopolymers, and carriage structure. The carriage structure includes a means for either moving the source of electromagnetic radiation over soil in situ or moving the soil so that the soil is exposed to the source of electromagnetic radiation. A radiation shield is provided in some embodiments to absorb radiation and to prevent leakage to undesired points. The device can include optional components such as one or more of a cooling system, a carriage operation station and sensor processor. The systems, methods and devices of the present invention are particularly useful for eliminating at least substantially all pests from an area of treatment prior to introduction of desired agricultural crops.

According to one aspect of the present invention, a method is provided for sterilizing soil. The method typically includes positioning at least one microwave frequency electromagnetic radiation generating element proximal a region of soil to be sterilized, wherein the generating element is tuned to generate microwave radiation at a frequency or range of frequencies sufficient to interact with carbon-carbon bonds. The method also typically includes generating microwave radiation at the frequency, or range of frequencies, for an amount of time and at a sufficient power to generate a microwave energy flux sufficient to render non-viable any organisms in the region to a specified depth.

According to another aspect of the present invention, a method is provided for removing pests from soil to a specified depth, in situ. The method typically includes exposing an area of soil to a sufficient energy flux, within a sufficiently short exposure time, of microwave frequency electromagnetic radiation at a frequency tuned to sufficiently damage all nucleic sequence information and biomacromolecules having information content Such that substantially all organisms are rendered non-viable in the area to the specified depth.

According to yet another aspect of the present invention, a method is provided for reconstituting an altered soil microbial balance resulting from a soil insult to a pre-alteration microbial balance. The method typically includes exposing an area of soil to a sufficient energy flux, within a sufficiently short exposure time, of microwave frequency electromagnetic radiation at a frequency tuned to sufficiently damage nucleic sequence information and biomacromolecules having information content so as to produce an area of treated soil that is substantially sterile to a specified depth. The method also typically includes inoculating the area of treated soil with one or more microbes to produce an inoculated area of treated soil. The inoculation reintroduces microbes required to reconstitute the pre-alteration microbial balance, and under ambient conditions the inoculated area of treated soil obtains a pre-alteration microbial balance at an earlier time than may be obtained by removing the soil insult.

According to yet a further aspect of the present invention, a device for in situ treatment of soil is provided. The device typically includes a source of microwave frequency electromagnetic radiation, and a carriage. The carriage is configured to move the source of electromagnetic radiation to expose the soil to a sufficient radiation energy flux within a sufficiently short exposure time to a specified depth so as to sufficiently damage all nucleic sequence information such that substantially all organisms contained in the soil are rendered non-viable to the specified depth.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1 depicts a side view for a device mounted on a carriage with a radiation shield, and rollers to control the depth of the device, the array deck having five rows of four microwave frequency electromagnetic radiation emitting elements, according to one embodiment.

FIG. 2 depicts the front view of the array of FIG. 1 without the guide rollers.

FIG. 3 depicts the layout of a single microwave frequency electromagnetic energy source according to one embodiment.

### DETAILED DESCRIPTION OF THE INVENTION

Devices, systems and methods are provided for disrupting biopolymers such as fats, carbohydrates, and other biomacromolecules that contain nucleic sequence information using contained microwave energy. A device according to the present invention includes a source of microwave electromagnetic radiation tuned to a frequency that disrupts one or more biopolymers in a pest organism. The device also typically includes a carriage and a radiation shield to prevent leakage of radiation in a direction other than the intended direction of treatment. The radiation shield generally includes a radio opaque barrier such as a RF reflective shield, magnetic curtain and optionally carbon foam. The biopolymers that are the target of the radiation typically are those associated with pests such as pathogenic organisms, including fungi and bacteria inhabiting the rhizosphere, undesirable plant matter such as seeds and weeds, insects, nematodes, microbes and the like that are detrimental to agricultural cropping systems and crops. Generally, exposure of the biopolymers to treatment is carried out in situ by moving a source of the contained microwave energy over an area to be treated but also can be carried out by moving the material to be decontaminated to within the range of the energy source. As an example, contaminated material, such as acreage intended for cultivation of crops, is treated by exposing the soil to a predetermined depth to a sufficient energy flux of microwave frequency electromagnetic radiation at a frequency tuned to dissociate biopolymers for a sufficient period of time so as to eliminate substantially all pests and/or contaminating material in the area of treatment. The frequency to which the energy source is tuned generally is about 2.4 to 2.6 GHz, however this does not exclude other frequencies in the ISM band, or other bands, e.g., L-band, S-band, etc., which may be employed, especially in the 1-20 GHz range determined by the resonance of the chemical bonds being broken within the cellular structure of the specific pest that is to be destroyed. For example, in one embodiment, it is preferred that the source(s) be tuned to generate a frequency of between about 2.48 GHz and about 2.52 GHz, and more preferably about 2.50 GHz, to interact with single carbon-carbon bonds. With sufficient energy flux as described herein such frequencies will easily break or destroy the single C-C bonds. Such frequencies also advantageously microfracture soil elements and assist with soil ionization and release of greenhouse gases. The source(s) may be tuned to other frequencies to target other chemical bonds and structures as desired. For example, a source can be tuned to about 2.45 GHz to interact with water molecules (e.g., heat), or a source can be tuned or configured to generate a frequency capable of interacting with higher level carbon bonds, e.g., triple carbon bond at 2800 GHz. Additionally, sources in an array of sources may be tuned to different frequencies.

In preferred aspects, the dwell time generally varies from about 0.5 to 5 seconds or more depending upon variables such as the type of soil (e.g., constituents and density), the amount of water in the soil, and the predetermined depth of soil to be treated. The treatment kills substantially all microorganisms in the area of treatment, including organisms beneficial to the growth of crops. Depending upon the type of crop to be planted it may therefore be necessary following treatment of an area to inoculate the treated area with beneficial organisms to provide a desired soil microbial balance. The inoculation can be by direct introduction of one or more microbes into the soil prior to planting, and/or the desirable microbes can be introduced by way of coatings on the seed to be planted and/or included with soil adhering to the roots of transplanted seedlings.

The present invention offers several advantages over existing technologies for removing pests from soil. As an example, using the present invention, substantially all biological material, including eukaryotic, prokaryotic and viral microbes, weeds, undesirable seeds, fungal spores, nematodes, and even insects are killed with a single treatment. Although some desirable insects and annelids may be removed from the soil by the treatment, such species are typically absent or economically irrelevant to most commercial agricultural production, and can be re-populated post treatment as desired. As an additional aid in re-establishing desirable microbes in the soil, the surface of minerals present in the soil may be micro-fractured by the microwave treatment making them easier for the microbes to digest.

Other advantages of the present invention include the absence of toxic residue or after-effects of the treatment other than quickly dissipated dry heating of the soil. Hence, no ground water or soil contamination, or chemical odors are possible using the present invention. Another advantage over most chemical soil treatment methods is that the microwave treatment of the present invention allows for essentially immediate cultivation of the soil after treatment. Furthermore, unlike treatments with chemicals such as methyl bromide which require covering the treated area to maintain the chemicals in the soil, which can trap subterranean living animals under the covers during the application of the toxic gas, the relatively slow moving large machinery for in situ soil treatment is more likely to drive most non-target animals in the ground near its path away before they arc subjected to the radiation. As an additional advantage, the soil preferably is not tilled prior to treatment, avoiding undesirable mixing of the topsoil with underlying strata and reducing costs from repetitive working of the soil.

According to one embodiment, a soil treatment device includes at least one source of microwave frequency electromagnetic radiation. The scale of the device may be relatively small, for example utilizing a single 30 kW element as a microwave source, in which case, with the carriage fixed in place, a flux is delivered to an area, for example, of less than about 10 square feet. Most commercial agricultural applications will require a larger device, for example, preferably an array of at least two to five microwave sources, more preferably an array of at least four to ten microwave sources, and most preferably an array of ten or more. In preferred aspects, each microwave radiation emitting element has a power output sufficient to achieve the desired flux. In one embodiment, for example, each element has a power output of at least about 30 kilowatts, which is generally sufficient for irradiating soil to a depth of about 4 feet or more. It should be appreciated that lower power emitting or higher power emitting elements can be used, depending primarily on the desired power flux. The flux from such an array can be used to treat an area of 100 square feet or more.

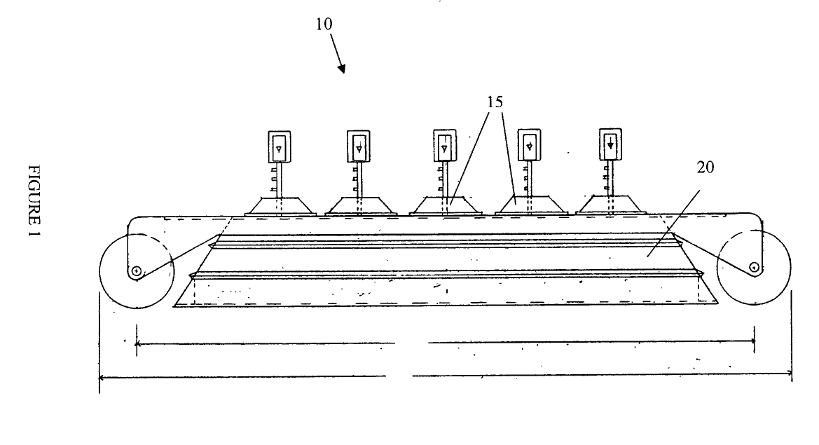

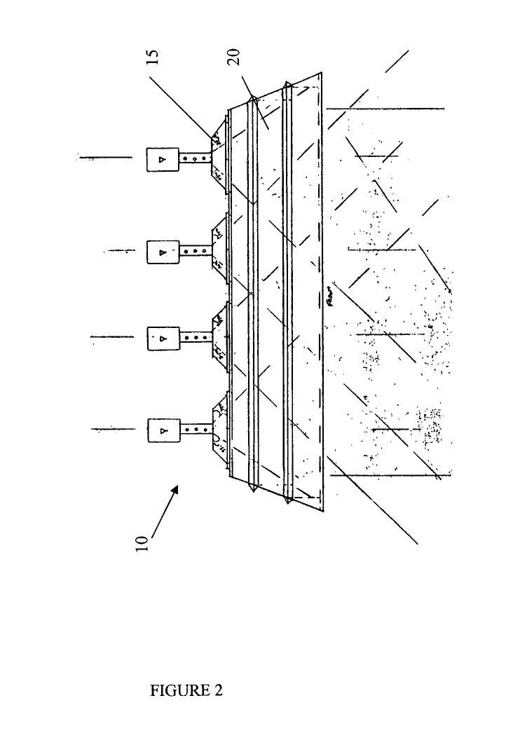

FIGS. 1 and 2 illustrate side and front views, respectively, of a treatment device 10 according to one embodiment. Treatment device 10, as shown includes an array of twenty microwave frequency electromagnetic radiation element 15 arranged in five rows of four each row. A metal cover on the array can be used to help redirect energy into the ground. The frequency of microwave radiation employed in the device is in a range appropriate for sterilization of the soil to which it is delivered. Thus the microwave frequency electromagnetic radiation is preferably at a frequency between about 2.4 GHz to about 2.6 GHz, more preferably between about 2.45 GHz to 2.55 GHz, and even more preferably at a frequency between about 2.48 GHz to 2.52 GHz.

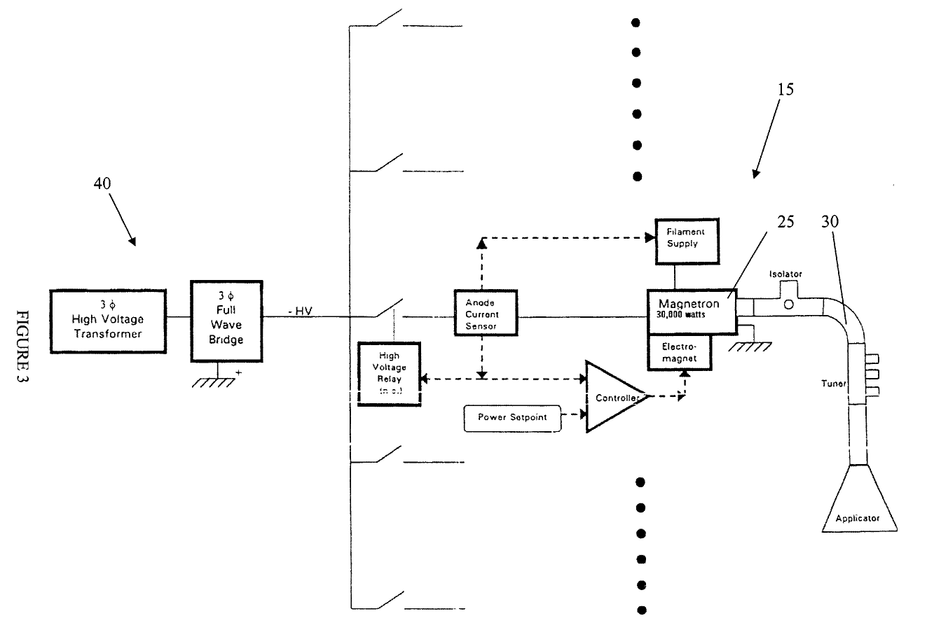

FIG. 3 illustrates a schematic of a microwave frequency electromagnetic radiation element 15 according to one embodiment. Microwave frequency electromagnetic radiation element 15 is coupled via a high voltage relay to a high voltage power source 40, such as a gas powered generator, including a transformer and full wave bridge as is well known. Radiation element 15 includes a magnetron 25 designed and tuned to emit microwave radiation of a desired frequency, or frequency range. For example, in one embodiment magnetron 25 emits radiation having a frequency of approximately 2.5 GHz with a power of approximately 30 kilowatts (continuous). The magnetron 25 may be spiked to produce power of approximately 45 kilowatts if desired. One useful magnetron is the CWM-30S magnetron, which provides a continuous power output of approximately 30 kilowatts at a frequency of approximately 2.45 GHz, supplied by California Tube Laboratory of Watsonville, California (website: www-dot-caltubelab-dot-com). The CWM-30S may be customized to produce a frequency output centered at approximately 2.50 GHz by optimizing the cooling in the impedance cavity, preferably upon manufacture of the anode magnetron tube. Microwave frequency electromagnetic radiation element 15 also includes an applicator guide tube 30 designed and configured to guide electromagnetic radiation to be delivered through the applicator end. A cooling system, such as a water jacket-pump assembly, may be implemented to provide cooling to radiation element 15, including tube 30 and magnetron 25.

In order to prevent radiation exposure to other than the area to be treated, the device 10 in one embodiment includes a radiation shield 20. The shield 20 preferably includes one or more barriers to microwave frequency electromagnetic radiation matter and/or material barriers, and/or electromagnetic field barriers. Generally, the barrier includes at least a matter barrier such as a flexible mesh or chain-link made of a material opaque to microwave frequency electromagnetic radiation. Examples of materials suitable for use as barriers include metals and graphite, and an appropriate barrier may comprise a metallic mesh overlaid with a carbon/graphite containing foam. Alternatively or in addition, an electromagnetic field barrier may be employed, the electromagnetic field barrier comprising a plurality of electromagnetic fields, typically magnetic or B fields, oriented to contain the microwave frequency electromagnetic radiation both laterally and vertically. Although the barrier may be absorptive or reflective, preferably, for increased power utilization efficiency, it is reflective and oriented such that some of the microwave frequency electromagnetic radiation impinging on the barrier is reflected downwards into the soil. To minimize leakage of radiation on uneven ground, in one embodiment a flexible magnetic curtain is mounted on at least the front and preferably the back and/or sides of the carriage. Because surface irregularities will always exist, microwave radiation detectors are preferably placed laterally to, e.g. just outside, the containment area at the ground level to detect and monitor any radiation leakage.

The device in one embodiment includes a means for either moving the source of electromagnetic radiation over soil in situ or moving the soil so that the soil is exposed to the source of electromagnetic radiation. Typically the moving means is a carriage that includes a drive train operably joined to a mechanical assembly that includes a weight bearing part such as one or more of a wheel, a cog and a track. When the weight bearing part is a cog, the carriage typically moves in a path determined by a guide such as an overhead or underlying rail or wire on which the cog rides. Preferably the carriage comprises a steer able motor vehicle riding on wheels, tracks or a combination thereof. Tracks and their assemblies provide increased traction while potentially augmenting lateral radiation shielding.

A device of the present invention optionally incorporates a cooling system that maintains the device at a sufficiently cool temperature to permit safe operation and adequate durability of the materials used in components of the device. Considerable surface heat, and high surface temperatures, results from microwave absorption as a result of application of the magnitude of power required to penetrate the soil sufficiently for typically desired sterilization depths. Although any cooling method may be employed including active heat pumping or a refrigeration cycle based cooling system, a water based cooling system is preferred. Water has a high heat capacity and is consequently an excellent “passive” coolant. A cooling system that finds use with the device includes a waterjacket containing circulating water, either pumped or circulating by virtue of source water pressure, wherein cold water influx and hot water efflux is by one or more hoses. Preferably the cooling system is a closed loop system.

The soil sterilization device may optionally include a carriage operation station, from which the carriage is driven across the soil and the sterilization process is controlled and various instruments provide information to a human operator. Preferably the instrumentation connects sensors or sensor/actuators to a processor, which provides an output to one of, or preferably to both, a processor control interface and a graphical user interface. In embodiments having the processor linked to both a processor control user interface and a graphical interface in the carriage operation station, the operational controls are controllable by a processor via the processor control interface, with operator override, or manually by an operator, with the benefit of both instrument readings and processor generated information. As an example, because a high power microwave source is employed to deliver enough energy at the desired depth, considerable heat is produced from microwave absorption. This may be especially problematic with pockets of organic material, such as buried peat, which may be ignited. Such pockets may absorb more energy and reflect less microwave energy upwards.

Another optional component of sterilization device of the present invention is a metal detector mounted, e.g., on the front of the device; metallic deposits in the soil will scatter the microwave energy and decrease the depth of energy penetration. A sonic signal to alert any animals in the area to be treated can also be incorporated into the device.

In operation, the sterilization device provides a sufficient energy flux to deliver a sufficient radiation power flux to a specified depth for a sufficiently long exposure time so as to substantially destroy pests contained in the soil to the specified depth. A sufficient energy flux within a sufficiently short exposure period helps overcome dissipation of absorbed energy, and is typically obtained by delivering a sufficient radiation power flux at a specified depth for a sufficiently long exposure time so as to substantially remove all pests contained in the soil to the specified depth. Because microwave radiation energy is both absorbed and reflected to some extent by the soil, power (and consequently energy flux for a given exposure time) is attenuated with depth, e.g. penetration is somewhat inversely proportional to depth and flux at any given depth is less than flux at the surface of the soil, e.g. the soil-air interface.

The depth of soil sterilization depends at least in part upon the specific desired outcome of treatment of the soil. When the purpose primarily is to remove pests such as seeds for weeds and other undesirable plants, then generally the specified soil depth for sterilization is at least about 0.50 feet, preferably about at least 0.5 to 1.5 feet. In most other contexts, sterilization is to a soil depth of at least about 1 foot, more preferably at least about 4 feet, yet more preferably at least about 6 feet, and even more preferably at least about 8 feet and most preferably to remove substantially all biological sequence information from the surface to a depth of greater than 9 feet. Ranges of practicable sterilization depths for the methods and devices of the invention include: between the surface and about 4 feet; preferably between the surface and about 6 feet; more preferably between the surface and about 8 feet. Typically, for sterilization to about six feet, sufficient soil surface radiation energy flux is at least about 9000 joules per square foot, but this energy flux must occur in a sufficiently brief exposure period, or dwell time, to overcome any dissipation of absorbed energy. Thus, adequate penetration to a specified depth is more easily expressed in terms of a minimum soil surface radiation power flux and adequate or sufficiently long exposure time to deliver enough energy, albeit quickly relative to dissipation time, to affect killing of substantially all pests. The device used therefore should deliver a soil surface radiation power flux of between at least about 1800 watts per square foot (W/ft²) and preferably at least about 3600 watts per square foot to a specified location for an adequate time to yield a soil surface radiation energy flux of between at least about 9000 joules per square foot, and preferably about 18,000 joules per square foot. For various soils, surface radiation energy flux magnitudes of at least about 1800 joules per square foot (J/ft²) to a soil surface radiation energy flux of at least about 18,000 joules per square foot may be obtained in between (e.g., the adequate time to yield a radiation energy flux of at least about 9000 joules per square foot,) about 0.5 seconds and about 5 seconds, preferably between about 1.5 seconds and about 3 seconds, more preferably between about 2 seconds and about 2.5 seconds. It should be noted that in actual practice these values may be increased or decreased depending on the soil moisture, chemistry, density, target depth of control, organic molecule target and other parameters specific to the treatment site. It should be further noted that as the power flux is increased (e.g., to about 4000 watts per square foot or more) the total energy input is decreased because the target molecules are more rapidly overloaded with energy and have significantly less time to dissipate the energy before destruction.

In one embodiment, a device of the present invention includes only a single radiation source. Such device may be used as a mobile hand carried or carriage mounted treatment device for treating, e.g., about 1 to 4 square feet for each dwell time interval.

Typically a device of the present invention includes an array of two to twenty or more radiation sources mounted on a carriage that can be used to expose from about 4 square feet to about 160 square feet with the carriage fixed, and depending upon precise geometry, can after treating the initial area of exposure, be moved, e.g., at under about five miles an hour, to achieve significant sized areas of treatment per work day to depths of six feet or greater. For example, returning to FIGS. 1 and 2, for a 10 foot by 12.5 foot array source with twenty 30 kW elements delivery of a surface energy flux of 9000 joules/ft² at surface power flux of 4800 watts/ft² can be achieved with an exposure or dwell time of about 1.9 seconds. This flux could be provided over a large area, for example, after remaining stationary for 1.9 seconds, by traveling at about 6.5 ft/s, or about 4.4 miles per hour.

The soil to be treated generally should be tested prior to treatment to determine if it is suitable for treatment. Penetration through any given layer of soil will vary depending on soil inorganic makeup and stratification, water content and organic content. Thus, the required exposure or dwell time for a given microwave frequency electromagnetic radiation source to achieve a desired result will vary depending on these factors. Generally, the soil should contain less than about 10% organic matter. As penetration is reduced by water content, the soil is preferably dry to the touch. Thus, the water content preferably is below the permanent wilting point, generally less than about 20% of field capacity, preferably less than 10%, more preferably in a range of 3-9%. Generally, soils with a heavy clay content such as adobe are not amenable to treatment unless dry. Unlike other soil treatments, the soil preferably is not tilled prior to treatment as this can create dust, traction and rut-formation problems. Only the smoothing out of surface irregularities is desired.

An artisan of ordinary skill in the art will apprehend that routine calibration experiments can be performed to measure penetration of microwave radiation, for example, by measuring microwave power and energy flux, and direct killing of microbes at various depths, by either boring under the device in situ for placement of the probe or microbe assay, simulating exposure in the laboratory using transported bored soil samples or simulating the soil to a depth based on bore data. Laboratory calibration experiments have the advantage of permitting simulation of the effect of changed conditions, such as encountering an area above an underground stream where moisture content of the soil is increased. Such data for various locations can be developed into a database for future reference for the calibrated locale and to computationally predict calibration of nearby or geologically homologous plots.

A device of the present invention is preferably used on relatively even terrain to minimize the risk of radiation leakage. Furthermore, because large planar arrays of emitters do not lend themselves to uneven terrain, traction problems for heavy machinery on large inclines, and radiation containment problems created by surface irregularities and roughness, a large scale machine should not be used on extremely rough ground or a substantial incline. Smaller scale machines may be useful in such circumstances for spot by spot treatment.

An artisan of ordinary skill in agricultural soil treatment and fumigation will appreciate that the treatment is preferably tailored to both the problem and the soil type and conditions in the locale to be treated, or soil microenvironment for optimum results. The invention will be illustrated in more detail with reference to the following Examples, but it should be understood that the present invention is not deemed to be limited thereto.

### EXAMPLES

### Example 1

### Soil Calibration

From a locale where sterilization to 6 feet is desired, a trench is excavated to 8 feet deep. A single 30 kW microwave emitter is placed over the soil along side the trench cloaked with a shield of the type intended to be employed in the field. The element covers 6.25 ft², the same coverage per element as in a 100 square foot 16 30 kW element array. At 3 foot and 6 foot depths, microwave energy and power flux detectors and heat detectors are inserted. Also at these depths small holes are drilled for insertion of small vials containing biological material such as, for example, replicating cellular microbes, spores, virus particles, and seeds. These vials of glass or other material transparent or sufficiently translucent to the frequency of microwave radiation employed. A bacterial pathogen is believed to be causing the problem at the locale to be treated. Calibration tests on bacteria placed in vials at 3 and 6 foot depths confirm killing of all tested species at both depths at ambient soil conditions and with added water content corresponding to a doubling of ambient content. The microwave penetration as measured by energy and power flux is adequate, although increased water is shown to affect penetration somewhat increasing attenuation over surface flux.

### Example 2

### Post Sterilization Soil Amendments

The treatment does not leave toxic residues; therefore both pathogenic and nonpathogenic microorganisms will begin to repopulate the treated area after treatment. But unassisted natural reintroduction of beneficial organisms may not occur in a sufficiently short period for the desired agricultural applications. Organisms must be reestablished in the soil after treatment to maximize the productivity of the soil. Thus commercial inoculums and other products, such as Arouse™, may be added to the soil at a rate according to the manufacturer's directions to maximize productivity of the soil post treatment.

All patent and literature references cited herein are incorporated herein in their entireties as if each individual publication or patent application were specifically and individually indicated to be incorporated by reference.

While the invention has been described by way of example and in terms of the specific embodiments, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A method of sterilizing soil, comprising:
positioning at least one microwave frequency electromagnetic radiation generating element proximal a region of soil to be sterilized, said generating element being tuned to generate microwave radiation at a frequency sufficient to interact with carbon-carbon bonds; and
generating microwave radiation at said frequency for an amount of time and at a sufficient power to generate a microwave energy flux sufficient to render non-viable any organisms in said region to a specified depth.

2. The method of claim 1, wherein the specified depth is between about 1 foot and about 4 feet.

3. The method of claim 1, wherein the specified depth is greater than about 4 feet.

4. The method of claim 1, wherein said generating element has a power output of at least about 30 kilowatts.

5. The method of claim 4, wherein the amount of time is between about 0.5 seconds and about 5 seconds.

6. The method of claim 4, wherein the amount of time is greater than about 5 seconds.

7. The method of claim 1, wherein the sufficient microwave energy flux is at least about 9000 joules per square foot.

8. The method of claim 1, wherein positioning includes mounting the at least one microwave frequency electromagnetic radiation generating element on a carriage stricture, and moving said carriage structure over said region of soil.

9. The method of claim 1, wherein said at least one microwave frequency electromagnetic radiation generating element is stationary, and wherein positioning includes moving said region of soil relative to said stationary radiation generating element.

10. The method of claim 1, wherein said frequency is between about 2.48 GHz and about 2.52 GHz.

11. A method of removing pests from soil to a specified depth, in situ, the method comprising exposing an area of soil to a sufficient energy flux, within a sufficiently short exposure time, of microwave frequency electromagnetic radiation at a frequency tuned to sufficiently damage all nucleic sequence information and biomacromolecules having information content such that substantially all organisms are rendered non-viable in said area to the specified depth.

12. The method of claim 11, wherein the specified depth of soil treatment is about 0.5 feet to about 4 feet.

13. The method of claim 11, wherein the specified depth of soil treatment is greater than about 4 feet.

14. The method of claim 11, wherein the pests include one or more of microbes, weeds, seeds, nematodes, and insects in the soil.

15. The method of claim 11, wherein exposing includes applying a minimum power flux of microwave frequency electromagnetic radiation of at least about 1800 watts per square foot to the surface of the soil.

16. The method of claim 11, wherein the source of microwave frequency electromagnetic radiation comprises one of a single microwave emitting element and an array of microwave emitting elements.

17. The method of claim 16, wherein each microwave radiation emitting element has a power output of at least about 30 kilowatts.

18. The method of claim 16, wherein the microwave frequency electromagnetic radiation is applied to the surface of said area for between about 0.5 seconds and about 5 seconds so as to provide a soil surface radiation energy flux of at least about 9000 joules per square foot.

19. A method of reconstituting an altered soil microbial balance resulting from a soil insult to a pre-alteration microbial balance, comprising:
exposing an area of soil to sufficient energy flux, within a sufficiently short exposure time, of microwave frequency electromagnetic radiation at a frequency tuned to sufficiently damage nucleic sequence information and biomacromolecules having information content so as to produce an area of treated soil that is substantially sterile to a specified depth; and
inoculating the area of treated soil with one or more microbes to produce an inoculated area of treated soil,
wherein the inoculation reintroduces microbes required to reconstitute the pre-alteration microbial balance; and
wherein under ambient conditions the inoculated area of treated soil obtains a pre-alteration microbial balance at an earlier time than may be obtained by removing the soil insult.

20. The method of claim 20, wherein the specified depth of soil treatment is about ½ foot to about 4 feet.

21. The method of claim 20, wherein the specified depth of soil treatment is greater than about 4 feet.

22. The method of claim 20, wherein a soil surface radiation power flux of at least 1800 watts per square foot is delivered to the area of soil for an adequate time to yield a soil surface radiation energy flux of at least about 9000 joules per square foot.

23. A method of altering soil microbial balance to a desired microbial balance, comprising:
removing from the soil to a specified depth in situ substantially all biological information by a treatment comprising exposing the specified depth of soil to sufficient energy flux, within a sufficiently short exposure time, of microwave frequency electromagnetic radiation at a frequency tuned to dissociate biomacromolecules having information content to produce an area of treated soil that is substantially sterile to the specified depth; and
inoculating the area of treated soil with one or more microbes to produce an inoculated area of treated soil, wherein the inoculation introduces microbes required to constitute the desired microbial balance; and
wherein under ambient conditions the inoculated area of treated soil obtains the desired microbial balance.

24. A device for in situ treatment of soil comprising:
a source of microwave frequency electromagnetic radiation; and
a carriage;
wherein the carriage is configured to move the source of electromagnetic radiation to expose the soil to a sufficient radiation energy flux within a sufficiently short exposure time to a specified depth so as to sufficiently damage all nucleic sequence information such that substantially all organisms contained in the soil are rendered non-viable to the specified depth.

25. The device of claim 24, further comprising a radiation shield configured to prevent leakage of radiation to undesired areas.

26. The device of claim 24, wherein the microwave frequency electromagnetic radiation generated by the source has a frequency between about 2.4 GHz to about 2.6 GHz.

27. The device of claim 24, wherein the microwave frequency electromagnetic radiation generated by the source has a frequency between about 2.48 GHz to about 2.52 GHz.

28. The device of claim 24, wherein the source includes one of a single microwave radiation emitting element and an array of microwave radiation emitting elements.

29. The device of claim 28, wherein each microwave radiation emitting element has a power output of at least 30 kilowatts.

30. The device of claim 24, further including a radiation shield comprising one of a matter barrier and an electromagnetic field barrier.

31. The device of claim 24, further including a cooling system configured to maintain the device at a sufficiently cool temperature during operation.

32. The device of claim 31, wherein the cooling system comprises a water jacket and a pump for circulating water in the water jacket.

33. The device of claim 24, wherein the carriage comprises a drive train operably linked to a mechanical assembly comprising a weight bearing portion selected from the group consisting of one or more wheels, a cog and a track.

34. The device of claim 33, wherein the weight bearing portion is a cog, and the carriage moves in a path determined by a rail or wire.

35. The device of claim 24, wherein the carriage comprises a steer able motor vehicle riding on wheels, tracks or a combination thereof.

36. The device of claim 24, wherein when the carriage is fixed in place, a radiation flux is delivered to an area of up to about 160 square feet.

37. A device for in situ treatment of soil comprising:
a source of microwave frequency electromagnetic radiation;
a carriage;
a radiation shield; and
a cooling system
wherein the carriage moves the source of microwave frequency electromagnetic radiation to expose the soil to a sufficient radiation flux to a specified depth for a sufficient time to destroy or render non-viable all nucleic sequence information contained in the soil to the specified depth, wherein the radiation shield prevents leakage of radiation to undesired points, and wherein the cooling system maintains the device at a sufficiently cool temperature to permit safe operation and adequate durability of materials used in components of the device.

38. The device of claim 37, further comprising a carriage operation station for controlling movement of the carriage.

39. The device of claim 38, wherein the carriage operation station includes operational controls and a processor control interface, the operational controls controllable by a processor or manually by an operator.

40. The device of claim 38, further comprising a processor with input from sensors and output to one or both of the processor control interface and a graphical interface, wherein the carriage being operated from the carriage operation station by an operator manually or by the processor with operator override, and wherein the processor control interface provides the operator with information from the processor.
